(19) United States
(12) Patent Application Publication
(10) Pub. No.: US 2026/0278186 A1
(43) Pub. Date: Sep. 17, 2026

(54) A METHOD AND SYSTEM FOR DESIGNING AND DISPLAYING VIRTUAL MODELS OF CUSTOM EAR DEVICES

(71) Applicant: 3Shape A/S, Copenhagen K (DK)

(72) Inventors: Søren Hegelund MAAGAARD, Copenhagen K (DK); Rasmus KONGSKOV, Copenhagen K (DK); Jakob MØLLER-ANDERSEN, Copenhagen K (DK)

(73) Assignee: 3Shape A/S, Copenhagen K (DK)

(21) Appl. No.: 19/167,618

(22) PCT Filed: Mar. 26, 2024
(86) PCT No.: PCT/EP2024/058115

§ 371 Date: Sep. 22, 2025

(30) Foreign Application Priority Data
Mar. 31, 2023 (EP) 23165919

(51) Int. Cl. G06F 30/12; G06F 30/27; G06F 111/04; G06F 111/16; G06F 111/20; G06F 113/10; G06F 119/18

(52) U.S. Cl. CPC G06F 30/12; G06F 30/27; G06F 2111/04; G06F 2111/16; G06F 2111/20; G06F 2113/10; G06F 2119/18

(57) ABSTRACT

A method for designing virtual models of custom ear devices according to user input and component and/or material information associated with components and/or materials of a target product is provided. The method may include steps for displaying the virtual models together with meta data that include information on the device's compatibility with the configuration requirements and the contained components. A computer-readable storage medium or computer program, including commands, which when executed by a computer, causes the computer to execute and perform the method, is also provided. A system for determining virtual models of custom ear devices is further disclosed. The system may further be configured to display the virtual models of custom ear devices together with associated meta data on a graphical user interface.

### FIELD

The disclosure relates in general to automatic product suggestion of custom ear devices, and in particular, to design virtual models of custom ear devices based on a combination of input requirements and component information and display the designed virtual models together with relevant meta data on a graphical user interface.

### BACKGROUND

The process of designing and manufacturing custom ear devices comprises practices that require an extensive amount of time and transportation. These practices include sending a physical impression mold of a patient's ear by mail to a manufacturing site where a custom ear device will be produced. Furthermore, dispensers (i.e., hearing aid professionals who advise patients and recommend hearing instruments) or other custom ear device professionals (e.g., producers or resellers of in-ear-monitors, custom earphones etc.), select a custom ear device model, electronic instrumentation, and optional components on the device, without any prior knowledge as to whether the preferred selection can be physically assembled into the resulting product (custom ear device). With these limitations, the dispenser or other custom ear device professional, is repeatedly contacted during the design of the instrument when physical component size limitations, or other compatibility issues, arise. This leads to long turnaround time for creating a patient's custom ear devices.

### SUMMARY

The invention is set out in the appended set of claims.

An aspect of the present disclosure is to allow for a designing of one or more virtual models of custom ear devices according to configuration requirements from a user input and according to component and/or material information that are associated with components and/or materials of a target product (i.e. the custom ear devices).

A further aspect of the present disclosure is to allow for displaying the designed one or more virtual models of custom ear devices together with meta data that comprise information on the designed one or more custom ear device's compatibility with the configuration requirements and the contained components (e.g., size, geometry, retention in the ear, etc.). Thus, an aspect of the present disclosure is to provide suggestions of custom ear devices in the form of one or more virtual models, so that time and steps spent on designing, manufacturing, logistics, and receiving custom ear devices, may be reduced.

According to the aspect, a computer-implemented method is provided for designing one or more virtual models of custom ear devices.

A computer-implemented method may be a method that may be performed by a computer, that may comprise one or more processors that is configured to execute one or more instructions, that when executed on a computer, performs the method.

Virtual models may be three-dimensional (3D) digital representations of three-dimensional (3D) physical models.

Custom ear devices may be custom hearing aid devices, custom in-ear monitors, custom earphones, custom ear molds, custom earshells, noise protection (e.g. for concerts, hunting, shooting, motor sports, etc.), custom sleep plugs, custom swim plugs, custom communication ear devices, custom radio earpieces (e.g. for news casters, security guards, etc.), or other similar types of custom ear devices having similar properties as the mentioned, which are to be designed based on a custom ear impression of a patient. Custom ear devices may be an invisible-in-the-canal (IIC) hearing aid, completely-in-the-canal (CIC) hearing aid, in-the-canal (ITC) hearing aid, half-shell (HS) hearing aid, or a full shell hearing aid (FS)

Custom ear devices may comprise one or more of: custom hearing aid devices, custom in-ear monitors, custom earphones, or custom ear molds.

The expression “custom” may be understood as relating to a custom-built part (e.g., earphone) that is built to fit to a specific counterpart (e.g. a specific human ear) or for a specific purpose (e.g. the specific need of a specific person). For example, a custom ear device may be an ear device (e.g., a hearing aid device), that is specifically built to fit to an ear of a specific person.

The method may comprise a step of loading into one or more processors ear impression data.

The one or more processors may comprise one processor, such as a CPU (central processing unit), with one or more processor cores. The one or more processors may comprise more than one processor, such as a plurality of CPUs, such as a processing cluster, wherein each of the plurality of CPUs includes one or more processor cores. The one or more processors may be located locally on a desktop computer, a laptop, a smartphone, a tablet, a smart tv, or a scanner, such as a handheld scanner. The one or more processors may partly or fully be located locally in one or more of the above-mentioned devices. The one or more processors may partly or fully be located in a server or a remote computer, that may be remotely relative to where the design and/or displaying of the one or more virtual models of custom ear devices is performed. The one or more processors may fully or partly be part of a cloud-computing service and may thus allow for using remote resources or may allow for a cloud-based processing.

The one or more processors may thus for example all or partly be located inside a desktop computer, or may all or partly be located remotely in a server as a cloud-based computing and operably connected to the desktop computer by cable, by a wireless network through a router, or Internet connection. The one or more processors may be distributed between two or more of the above mentioned locations. For example, some of the one or more processors may be located in the desktop computer in the form of a CPU (central processing unit), while some other of the one or more processors may be located in a remote server in a “cloud” as a cloud based computing service, and connected to the desktop computer via the internet.

The ear impression data may be a virtual 3D model of an ear or part of an ear that has been scanned. The virtual 3D model may be obtained by scanning an ear or part of an ear using a handheld ear-scanner or by scanning an ear impression using a lab-scanner. The ear impression data may be 3D data, such as a 3D point cloud, or other scan data.

The ear impression data may be loaded into the one or more processors by performing a direct scan using a handheld ear scanner or a lab-scanner, by loading from a memory unit, by loading from a file, by downloading from the internet, etc.

For example, an audiologist may take a physical ear impression of a person with hearing loss. The physical ear impression may subsequently be scanned using a lab-scanner, thus obtaining ear impression data. The obtained ear impression data may be used to create a virtual 3D model of the physical ear impression. The virtual 3D model may be created using a processor that is operably connected to the lab-scanner (e.g. a processor in the lab-scanner, a processor that is in a desktop computer connected to the lab-scanner, or a cloud-based processor that is connected to the lab-scanner using the internet).

In another example, the obtained ear impression data may be received onto the audiologist's desktop computer (e.g. downloaded onto the audiologist's desktop computer from a server that is connected to the lab-scanner, received by e-mail, or otherwise received), and the virtual 3D model may be built on the audiologist's desktop computer, using a processor of the audiologist's desktop computer.

The method may comprise a step of loading into the one or more processors one or more configuration requirements that comprise one or more requirements for accepting a target product.

The configuration requirements may comprise one or more requirements for including, in a target product, one or more of: components, features, materials, size, retention, type, product, product series. The configuration requirements may usually relate to user preferences for a custom ear device.

The one or more configuration requirements may comprise one or more requirements for including, in a target product, one or more of: type of custom ear device (e.g. behind the ear hearing aid device, in the ear hearing aid device, in the ear monitor, earphones, etc.), a shell type, a product family (e.g. a group of products of the same type, brand, or performance), a specific product (e.g. a specific product of a specific brand of a manufacturer or reseller), a preferred manufacturer, a preferred product, product series or product family from a manufacturer, components (e.g. speaker, speaker size, microphone, amplifier, battery, battery capacity, etc.), material (e.g. natural rubber, plastic, silicone, textile, etc.), features (e.g. options such as Bluetooth connection, buttons, improved noise cancelling, remote control, charging case (e.g. custom charging case), wireless charging, etc.), dimensions (e.g. thickness, size, etc.), retention (e.g. type of retention such as coverage of the ear, retention by friction in the ear, retention by retention means such as a hook/anchor behind the ear, etc.), performance (e.g. hearing loss compensation, noise cancellation, power consumption, etc.), software (e.g. software features e.g. noise cancellation, speech enhancement, etc.), and/or information such as price, colour, or hearing compensation information (e.g. audiogram), and ventilation information (e.g., small, medium, or large).

The configuration requirements may be one of the above or a combination of two or more of the above-mentioned configuration requirements.

The above-mentioned configuration requirements may be requirements that a user may require for a target product to be selected and/or accepted, and may thus be sent as a manufacturing order to be physically manufactured by a custom ear device manufacturer.

The target product may be a virtual model of a custom ear device, a set of specifications that describe a custom ear device, or a physically manufactured custom ear device, that meets the requirements of the one or more configuration requirements. The target product may be a plurality of virtual models of custom ear devices, a set of specifications that describe a plurality of custom ear devices, or physically manufactured plurality of custom ear devices, that each meet the requirements of the one or more configuration requirements.

In an example, the target product may be a shell with impression of components on the inside surface of the shell. Designing such a shell according to the embodiment of the invention is advantageous as the obtained target product is manufacturable, accommodates for the components necessary to satisfy the one or more configuration requirements and fits the ear impression data thus fits the specific geometry of the ear where it will be mounted.

In an example, an audiologist, may in a graphical user interface of a software, displayed on the audiologist's desktop computer, select a set of configuration requirements comprising requests for a target product, based on a set of prerequisites of a person with hearing loss. The person with hearing loss may for example request a custom hearing aid that is of the type “in-the-ear hearing aid”, that is capable of compensating for a hearing loss of 20% of a normal human hearing, and that is capable of connecting to a smartphone using Bluetooth. The person with hearing loss may further require that the target product or an outer surface of the target product to be in a black colour, such that the target product is visually harder to detect when inserted inside the ear canal.

The method may comprise a step of providing access for the one or more processors to a datastore. The datastore may be a hard drive or a server. The datastore may be connected to servers, websites, or datastores, of one or more custom ear device manufacturers or manufacturers of components, materials, or software, for custom ear devices. The datastore may comprise a data storage such as a hard drive or a server, wherein data about products, components, features, materials, software, and information, from one or more ear device manufacturers are stored, and accessible by the one or more processors. The datastore may be a connection hub, providing or directing connection between the one or more processors and datastores or servers of one or more custom ear device manufacturers, wherein the datastores or servers of the custom ear device manufacturers comprise data or information of their respective products, components, features, materials, and software. The access may be provided to the datastore by a network connection such as the Internet. The one or more processors may for example access the datastore using an internet connection, for example by connecting to a server of the datastore using the internet.

The datastore may comprise component and/or material information that may be associated with one or more components and/or materials of the target product.

The components and/or materials may be products, such as hearing aid devices, custom hearing aid devices, in-ear monitors, custom in-ear monitors, earphones, custom earphones, ear molds, or custom ear molds; may be components, such as a part of the products. components of the products, microphones, speakers, amplifiers, microchips, batteries, Bluetooth connection, wireless antenna ; may be features, such as hearing loss compensation, noise cancellation, power consumption, speech enhancement, Bluetooth, wireless connection or communication, remote control; may be materials, such as polymeric materials, such as plastic, rubber, silicone, etc.; may be software, such as software that is configured to perform noise cancellation, speech enhancement, support wireless connection, support remote control, improve battery life, etc., and may be information related to the previously mentioned examples of components and/or materials, such as dimensions, properties, specifications, colour, price, etc.

The component and/or material information may be any information about, or data associated with, the components and/or materials. For example, the component and/or material information about the material and/or component “battery” may be type, brand, capacity, size, colour, which custom ear device it is compatible with, and/or price of the battery. Another example, the component and/or material information of a “product” may be: The amount of hearing loss the product can compensate, features (e.g. capable of Bluetooth connection), type of the retention (e.g. in-the-ear hearing aid), battery capacity, software that is included (e.g. comprising noise cancellation, speech enhancement, etc.), price, size, least venting hole size, etc.

The component and/or material information may comprise information that relates to one or more of: type of component, type of material, product, product series, features, component or material parameters, or performance.

The component and/or material information may comprise information associated with components and/or materials that may comprise geometric dimensions, such as lengths, widths, depths, and curvatures, and may further comprise virtual 3D models of the components and/or materials, and may further comprise information associated with properties such as material stiffness, temperature tolerances, etc.

The component and/or material information may be information and/or design rules that specify specific rules for designing virtual models of custom ear devices, such as e.g. that the manufacturer only allows 20 designs per order, that the manufacturer has specified a preferred prioritization for selecting a component or a product (e.g. for a specific product, material, or component, it is not allowed to print in a thickness that is less than 0.3 mm (millimeters), etc.).

The component and/or material information may comprise a fixed value. The fixed value may be any number. The component and/or material information may each, collectively, or in combination comprise a fixed value indicative of a fixed hierarchical order of the components and/or materials. The fixed value may be assigned to each individual component, material, component information, and/or material information in the datastore by the manufacturer.

For example, a fixed value may be assigned by a manufacturer to a particular product or a product information. A fixed value may be assigned to a particular product family or product series, thus the whole product family or product series having a fixed value collectively, or wherein each particular product in the product family or product series having the same fixed value.

The fixed value may be indicative of a fixed hierarchical order of the one or more components and/or materials.

The fixed hierarchical order may be an expression of a prioritization of the fixed value of each of the component and/or material information or to each of the one or more components and/or materials, according to their value. The fixed hierarchical order may be the first order of each one of the components and/or material information or each one of the one or more components and/or materials, expressed, ranked, arranged, or ordered systematically according to their value. The fixed hierarchical order may thus reflect the components and/or materials that are essential for a certain type of a custom ear device (with assigned highest values) and those components and/or materials that are optional for that type of the custom ear device (with assigned lower values).

The method may comprise a step of determining, using the one or more processors, one or more combinations between the component and/or material information from the datastore and the one or more configuration requirements. The obtained combinations represent manufacturable custom ear devices, each satisfying the one or more configuration requirements.

The one or more combinations may be a combination between each one of the component and/or material information from the datastore and each one of the one or more configuration requirements.

In a situation where no configuration requirement has been selected, the one or more combinations may be every custom ear device that is comprised of every one of the component and/or material information and every combination of every one of the component and/or material information.

In a situation where one or more configuration requirements have been selected, the one or more combinations may be every combination between each selected one or more configuration requirement and each component and/or material information. Thus, a first filtering may be performed, such that the custom ear devices may only be those who comprise component and/or material information that meet the requirements of the selected one or more configuration requirements.

Determining the one or more combinations may be performed by the one or more processors. The one or more processors may obtain information related to one or more selected configuration requirements, and subsequently access the datastore, and determine one or more combinations of each component and/or material information, that meet the requirements of the one or more configuration requirements, such that one or more custom ear devices may be designed that is comprised of every combination of the components and/or materials of the component and/or material information that meet the requirements of the configuration requirements. Thus, in an embodiment, the step of determining the one or more combinations is performed only for the component and/or material information that meet the one or more requirements of each of the one or more configuration requirements.

In an example, a configuration requirement may be specified wherein a custom ear device type is specified that is a hearing aid device which is capable of compensating a specific hearing loss, and which is capable of being manufactured in a specific size, that allows the hearing aid device to be retained in an ear canal of a specific person. The one or more processors may obtain information of the above mentioned specified configuration requirement, and subsequently access the datastore comprising the component and/or material information. The one or more processors may thus determine a set of combinations, wherein every possible custom ear device is determined that may be built using every component and/or material of the database that is of a type “hearing aid device”, that is capable of compensating the specified hearing loss, and that is capable of being manufactured in the specified size.

It is an advantage of the described embodiment that all manufacturable custom ear devices satisfying the user requirements are identified and presented to the user.

The step of determining the one or more combinations may comprise assigning a combined value of a combined hierarchical order to each of the one or more combinations. As an advantage, all manufacturable custom ear devices satisfying the user requirements may be ranked according to their fit to the one or more configuration requirements.

The combined value may be a combination of the respective fixed value of the component and/or material information. The combined value may thus be the respective fixed value of the component and/or material information. For example, if no configuration requirement is specified, or a configuration requirement is specified that doesn't comprise a value, then the combined value may be the fixed value of the respective component and/or material information that is used in the particular combination comprising the particular combined value.

The method may comprise a step of designing one or more virtual models of custom ear devices based on the one or more combinations and the combined value. In an embodiment, designing of the one or more virtual models of custom ear devices may comprise obtaining the one or more virtual models of custom ear devices from the datastore that are represented by the one or more combinations of components and/or material information and that satisfy the one or more configuration requirements. In a further embodiment designing of the one or more virtual models of custom ear devices may comprise generating the one or more virtual models of custom ear devices based on the ear impression data and the one or more configuration requirements.

Designing the one or more virtual models of custom ear devices may be performed by a design module. The design module may comprise one or more algorithms and/or one or more neural networks that are configured to design the one or more virtual models of custom ear devices based on an input from the one or more processors. The input from the one or more processors may be the one or more combinations or may be one or more instructions comprising information for designing the one or more virtual models of custom ear devices. The one or more processors may be configured to send a request to the design module for performing the design of the one or more virtual models of custom ear devices. The design module may be configured to receive a request from the one or more processor for designing the one or more virtual models of custom ear devices. The design module may be operably connected to the one or more processors. The design module may be a stand-alone software on a computer comprising the one or more processors, such as on an audiologist's desktop computer, or may be a software or a set of algorithms and/or neural networks located on a server, such as being a design service in the “cloud”.

Designing the one or more virtual models of custom ear devices may be based on the one or more combinations and the combined value. The one or more processors may be configured to send instructions to the design module comprising information related to designing the one or more virtual models of custom ear devices of the one or more combinations. The instructions may comprise the combined values, and the design module may be configured to design the one or more virtual models of the custom ear devices in a sequence that is according to the combined values. The design module may be configured to send the designed one or more virtual models of custom ear devices to the one or more processors or directly to a graphical user interface, such as a graphical user interface of a computer comprising the one or more processors.

The method may further comprise the step of selecting the target product from the one or more designed virtual models of custom ear devices such that the target product fits the ear impression data. In this way it may be ensured that, the manufacturable custom ear device, satisfying the one or more configuration requirements, also fits to the specific ear geometry of a patient. This selection may be performed by performing geometric comparison of the one or more designed virtual models of custom ear devices with a virtual model of the ear obtained from the ear impression data.

The method may further comprise the step of generating a control file for a 3D printer, wherein the control file comprises instructions for manufacturing of the target product. The control file may thus be a set of instructions for 3D printing of the shell with impression of components on the inside surface of the shell.

Another aspect of the disclosure is to provide a computer-implemented method for designing one or more virtual models of custom ear devices. The method may comprise loading into one or more processors ear impression data, loading into the one or more processors one or more configuration requirements that comprise one or more requirements for accepting a target product, providing access for the one or more processors to a datastore comprising component and/or material information associated with one or more components and/or materials of the target product, the component and/or material information comprising a fixed value indicative of a fixed hierarchical order of the one or more components and/or materials, determining, using the one or more processors, one or more combinations between the component and/or material information from the datastore and the one or more configuration requirements, and assigning a combined value of a combined hierarchical order to each of the one or more combinations, wherein the combined value may be a combination of the respective fixed value of the component and/or material information, and designing one or more virtual models of custom ear devices based on the one or more combinations and the combined value.

In an embodiment a computer-implemented method for designing one or more virtual models of custom ear devices is disclosed, the method comprising:
loading into the one or more processors ear impression data,
loading into the one or more processors the one or more configuration requirements that comprise the one or more requirements for accepting the target product,
providing access for the one or more processors to the datastore comprising component and/or material information associated with the one or more components and/or materials of the target product, the component and/or material information comprising the fixed value indicative of the fixed hierarchical order of the one or more components and/or materials,
determining, using the one or more processors, one or more combinations between the component and/or material information from the datastore and the one or more configuration requirements, and assigning the combined value of the combined hierarchical order to each of the one or more combinations, wherein the combined value comprises at least the respective fixed value of the component and/or material information,
designing the one or more virtual models of custom ear devices based on the one or more combinations and the combined value,
selecting the target product from the one or more designed virtual models of custom ear devices such that the target product fits the ear impression data, and
generating a digital file for production of the target product.

In an embodiment a computer-implemented method for designing one or more virtual models of custom ear devices is disclosed, the method comprising:
loading into one or more processors the ear impression data,
loading into the one or more processors one or more configuration requirements that comprise one or more requirements for accepting the target product,
providing access for the one or more processors to the datastore comprising component and/or material information associated with one or more components and/or materials of the target product, the component and/or material information comprising the fixed value indicative of the fixed hierarchical order of the one or more components and/or materials,
determining, using the one or more processors, one or more combinations between the component and/or material information from the datastore and the one or more configuration requirements, and assigning the combined value of the combined hierarchical order to each of the one or more combinations, wherein the combined value is a combination of the respective fixed values of the component and/or material information,
designing one or more virtual models of custom ear devices based on the one or more combinations and the combined value, and
generating the control file for the 3D printer, wherein the control file comprises instructions for manufacturing of the one or more virtual models of custom ear devices.

The design module is further described in details in a later section.

The one or more configuration requirements may each, collectively, or in combination comprise an optional value indicative of an optional hierarchical order of the one or more configuration requirements. The optional value may be a number. The optional value may be assigned to each individual requirement of the one or more configuration requirements, to a requirements of the one or more configuration requirements, or to one configuration requirement of the one or more configuration requirements, by a user, such as a person with hearing loss or an audiologist. The optional value may be assigned on a website, such as a website for ordering custom ear devices, on a software, or other interfaces for inputting data.

The optional hierarchical order may be an expression of a prioritization of the optional value each of the requirements of the one or more configuration requirements, or to one or more configuration requirements, according to their value. The optional hierarchical order may be the optional order of each requirement of the one or more configuration requirements, or of each one of the one or more configuration requirements, expressed, ranked, arranged, or ordered systematically according to their value.

In an example, an audiologist may, in a graphical user interface of a software displayed on the audiologist's desktop computer, select one or more configuration requirements, for example from a roll-down menu, and assign an optional value to each of the one or more configuration requirements.

In an example, an optional value may be assigned to each requirement in a configuration requirement individually. For example, the person with hearing loss may choose one configuration requirement that comprises a requirement wherein the type of custom hearing aid is an in-the-ear hearing aid, and may assign an optional value of 100 to that particular requirement, which may indicate a highest order or ranking in the hierarchy of the optional hierarchical order. The person with hearing loss may choose another requirement of the configuration requirement wherein the custom hearing aid is capable of connecting via a Bluetooth connection, and may assign an optional value of 90 to that particular requirement, indicating that the requirement of Bluetooth capability has a lower prioritization, order, or ranking, in the optional hierarchical order, than the requirement of the custom hearing aid being an in-the-ear hearing aid.

In another example, an optional value may be assigned to a set of requirements in a configuration requirement collectively. For example, the person with hearing loss may choose one configuration requirement that comprises a requirement wherein the type of custom hearing aid is an in-the-ear hearing aid, a requirement wherein the custom hearing aid is capable of connecting via a Bluetooth connection, and a requirement wherein a biodegradable material is used for an outer shell of the custom hearing aid. The person with hearing loss may subsequently assign an optional value of 100 to that particular configuration requirement, which may indicate a highest order or ranking in the hierarchy of the optional hierarchical order, and may thus indicate that this particular configuration requirement is the most preferred or most prioritized configuration requirement.

In an example where one or more configuration requirements have been selected, the one or more combinations may be assigned an optional value. An audiologist may for example select one or more configuration requirements on a graphical user interface, and assigning an optional value to each one of the one or more configuration requirements. The audiologist may for example select one configuration requirement wherein the custom ear device is of a type in-the-ear hearing aid, and assign an optional value of 100, and may select one configuration requirement wherein information is specified wherein a specific hearing loss is specified, and assign an optional value of 90, indicating that this configuration requirement is of a less priority than the configuration requirement comprising the optional value of 100. In another example, the audiologist may select a set of configuration requirements, and assign an optional value to the set of configuration requirements. The audiologist, may for example select a configuration requirement that the custom ear device is of a type in-the-ear hearing aid, may select a further configuration requirement of the type information, and select a specific hearing loss, and assign an optional value of 100 to the set of configuration requirement. The audiologist may select another set of configuration requirements, wherein the audiologist selects a configuration requirement wherein the type is of behind-the-ear hearing aid, a configuration requirement wherein the specific hearing loss is specified, and a configuration requirement wherein a feature is selected that allows for noise cancellation, and assign an optional value of 90 to the another set of configuration requirement, indicating that the another set of configuration requirement is of a less priority than the set of configuration requirement that has been assigned an optional value of 100.

In a situation where an optional value has been assigned to the one or more configuration requirements (i.e. the one or more configuration requirements comprising an optional value), the combined value may be a combination of the respective fixed value of the component and/or material information and the respective optional value of the one or more configuration requirements. Thus, the one or more configuration requirements may each comprise an optional value that may be indicative of an optional hierarchical order of the one or more configuration requirements. The combined value may combine the respective optional value and the respective fixed value.

The combined value may thus be a combination of the optional value of the one or more configuration information and the fixed value of the component and/or material information. For example, a combination between a configuration requirement comprising an optional value of 100 and a component and/or material information comprising a fixed value of 80, comprises thus a combined value of 180.

For example, the design module designs virtual models of custom ear devices according to a sequence ordered hierarchically according to a combined value of each combination of one or more combinations determined by the one or more processors, wherein the combined values combine the optional values and the fixed values of the respective one or more combinations. The design module may for example first design a combination having a combined value of 200, then a combination having a combined value of 150, then a combination having a combined value of 100, etc.

In yet another example, an optional value may be assigned to a configuration requirement based on a combination of individual optional values of each requirement of the configuration requirement. For example, a configuration requirement comprising a requirement having an optional value of 50 and another requirement having an optional value of 70, the configuration requirement may thus have an optional value of 120.

The method may comprise a step of evaluating the combined value for each of the one or more combinations and determining a designing sequence indicative of a hierarchically ordered sequence based on the combined value. The step of evaluating the combined value may performed by the one or more processors. The one or more processors may be configured to detect the value of the combined value.

The designing sequence may be one or more instructions for designing one or more virtual models of custom ear devices in a particular order. The instructions may be configured to instruct a design module to design the one or more virtual models of custom ear devices in a particular order. The designing sequence may comprise information related to the one or more combinations and related to the respective combined values of the one or more combinations. The particular order may be based on the combined values of the one or more combinations. The designing sequence may be determined by the one or more processors based on the combined values of the one or more combinations, by evaluating the combined values of the one or more combinations.

For example, a designing sequence may comprise a set of instructions for designing virtual models of custom ear devices according to a combination A with a combined value of 200, a combination B with a combined value of 180, and a combination C with a combined value of 150, in that particular order, wherein the combination with the highest combined value is to be designed first. A design module will subsequently design combination A, then combination B, and then combination C.

The method may further comprise a step of feeding into the design module the determined designing sequence. The one or more processors may be configured to feed the design module with the designing sequence, by sending the instructions of the design sequence to the design module. The design module may be configured to design the one or more virtual models of custom ear devices according to the designing sequence.

The method may further comprise a step wherein the design module is further configured to determine whether each designed one or more virtual models of custom ear devices, is accepted as a target product. Accepting the one or more virtual models of custom ear devices as target products may be based on geometrical constrains of the one or more virtual models of custom ear devices.

Determining whether a designed virtual model of custom ear devices may be accepted as a target product or not, may allow reducing the risk of building a custom ear device that does not fit to the components that are included in the custom ear device, or to the ear of the person who is to wear the custom ear device, such as the ear of a person with hearing loss.

Furthermore, determining whether a designed virtual model of custom ear devices may be accepted as a target product or not, may allow for the reduction of the number of designed virtual models of custom ear devices that are sent to the one or more processors or to a graphical user interface, and a filtering of the possible target product may thus be performed.

Determining whether a designed virtual model of custom ear devices may be accepted as a target product may be performed by the design module. The design module may be configured to design one or more virtual models of custom ear devices, and may further be configured to subsequently determine whether the designed one or more virtual models of custom ear devices may be accepted as target products.

Determining whether the designed one or more virtual models of custom ear devices may be accepted as target products may be based on geometric constraints. Thus in an embodiment, the method of the disclosure comprises selecting the target product from the one or more designed virtual models of custom ear devices with the ear impression data based on geometric constraints of the one or more virtual models of custom ear device.

The geometric constraints may be that parts of the one or more virtual models of custom ear devices may not intersect (go through) parts of a virtual 3D model of a physical ear impression of a person. The design module may further determine, based on geometric dimensions of the components and/or materials that are comprised in the virtual model of custom ear devices, whether the components and/or materials intersect with the designed one or more virtual models of custom ear devices or not. Geometric constraints may further be constraints for preventing or reducing magnetic interference between components, such that some components may not be positioned too close to each other and not arranged in a specific direction or orientation that may increase or allow magnetic interference.

The design module may further be configured to evaluate the designed one or more virtual models of custom ear devices, by determining whether each of the one or more virtual models of custom ear devices can virtually accommodate the one or more component and/or materials which they are associated with.

The method according to an embodiment comprises the step of selecting the target product from the one or more designed virtual models of custom ear devices such that the target product fits the ear impression data.

The method may further comprise a step of loading into the one or more processors the designed one or more virtual models of custom ear devices from the design module, and displaying the designed one or more virtual models of custom ear devices on a display.

For example, an audiologist selects some configuration requirements for accepting a target custom hearing aid device and assigns optional values to each of the configuration requirements in a graphical user interface of a software that is run on the audiologist's laptop computer. The processor of the laptop computer determines combinations of the configuration requirements and components and materials from a database comprising component and/or material information and thereto associated fixed values from several ear device manufacturers, related to the components and materials. The processor determines a combined value for each combination, based on the optional value of the respective configuration requirement of the particular combination and on the fixed value of the respective component and/or material information of the particular combination. The processor determines a designing sequence, comprising information about the configuration requirements and component and/or material information of each combination and their respective combined value, and with instructions to design virtual 3D models of each combination in an order beginning with the highest ranked combined value. The processor sends the designing sequence to a design module that is run as a design-service by a service provider on a server. The design module receives the designing sequence and builds a virtual model of a custom hearing aid device based on the combination from the designing sequence with the highest ranking combined value. The design module arranges the designed virtual model of the custom hearing aid inside the virtual 3D model of the ear impression, and determines whether the virtual model of the custom hearing aid may be arranged in the virtual 3D model of the ear impression without conflicting parts, such as parts from both virtual models intersecting. The design module further determines whether virtual 3D models of the relevant components and materials may be arranged inside the virtual model of the custom hearing aid device without conflicting parts, such as parts from the virtual model of custom hearing aid device and the virtual 3D models of the components and materials intersecting.

The design module subsequently sends the designed virtual model of the custom hearing aid device to the processor on the audiologist's laptop computer, with information relating to whether the were any conflicts or not. If there were no conflicts, the designed virtual model of the custom hearing aid may be accepted as a target custom hearing aid device. The design module subsequently performs the above mentioned process for the next combination in the designing sequence, with the second highest ranked combined value, and so on.

In another example, the design module only sends the designed virtual models of custom hearing aid devices to the processor of the audiologist's laptop computer, that are accepted as target custom hearing aid devices, discarding the designed virtual models of custom hearing aid devices that were not accepted as target custom hearing aid devices, thus performing a filtering of target custom hearing aid devices.

In another example, the design module only designs the virtual models of custom hearing aid devices according to the combinations and their respective combined values, and sends the designed virtual models of custom hearing aid devices to the processor of the audiologist's laptop computer. The processor subsequently determines whether each combination may be accepted as a target custom hearing aid device based on geometric constraints, in a similar process as described above.

For example, the design module may build one combination of a virtual model of custom ear device and determine a default venting hole. If the geometry of the virtual 3D model of the ear impression allows for a larger venting hole, the design module may determine a larger venting hole. If the geometry of the virtual 3D model of the ear impression does not allow for the default venting hole, the design module may determine a smaller venting hole. When the size of the venting hole is accepted such that not intersection between any part of the venting hole, the virtual 3D model of ear impression, or the virtual model of custom ear device occurs, the design module proceeds in determining a loudspeaker, etc. The final accepted designed virtual model of custom ear device may then be sent to the one or more processors to be displayed on a graphical user interface and made selectable as a target product by the user (e.g. audiologist or person with hearing loss).

In another embodiments, determining the sizes and preferences of components and materials for each combination may be performed be the one or more processors, and the information subsequently sent to the design module, wherein the design module builds the relevant virtual models and determines whether the relevant virtual models may be accepted as target products based on the geometric constraints, as described above. The design module subsequently sends the final accepted designed virtual models of custom ear devices to the one or more processors to be displayed on a graphical user interface and made selectable as a target product by the user (e.g. audiologist or person with hearing loss).

The method may comprise a step of automatically suggesting a product. The one or more processors may thus be configured to perform an automatic product suggestion based on the specified one or more configuration requirements and based on the component and/or material information for the database. The automatic product suggestion may be performed using a product compare engine that may use matrix comparison.

The method may comprise a step of determining, using the one or more processors, first meta data that may relate to the performance of the one or more components and/or materials of the target product.

The first meta data may be data that may be predetermined by the manufacturers of the custom ear devices or manufacturers of components and materials of custom ear devices, and that may relate to preferences, performance, and or specifications, associated with each custom ear device, product, component, and material, such as e.g. a specific product allows for a Bluetooth connection, allows for a small venting hole, allows for improved hearing, etc. First meta data that is associated with a component, such as a battery, may for example be battery capacity or that a battery allows for wireless recharging, etc. The performance of the one or more components and/or materials of the target product may be preferences, performance, and or specifications, associated with each custom ear device, product, component, and material. The first meta data may further comprise information about size, price, etc.

The first meta data may be located in the datastore, and each component and/or material information may comprise one or more associated first meta data.

The method may comprise a step of determining, using the design module, second meta data that may relate to the compatibility of the designed one or more virtual models of custom ear devices with components and in the ear.

The second meta data may be information that may be derived from the designed virtual models of the custom ear devices or from the designed virtual models of custom ear devices' interaction with the virtual 3D model of ear impression or virtual 3D models of components and or materials. The components may be components and materials of the component and/or material information from the datastore. The second meta data may further comprise information about retention in the ear, probability of occurring interference, size, placing, price, etc.

For example, the second meta data may be: improved retention, improved comfort inside the ear, etc.

Determining the second meta data may be performed by the design module. The design module may be configured to arrange each designed virtual model of custom ear devices into the virtual 3D model of the ear impression, and determine whether the arrangement is successful or acceptable, such that there is no intersection between any part of the virtual model of custom ear device and virtual 3D model of the ear impression.

The design module may further arrange each of the virtual 3D models of components and/or materials of the component and/or material information that are present in a specific custom ear device or that have been specified by a user (e.g. audiologist) to be part of the target product, inside each virtual model of custom ear devices, and determine whether the arrangement is successful or acceptable, such that there is no intersection between any part of the virtual model of custom ear device and virtual 3D model of the components and/or materials.

The method may comprise a step of loading into the one or more processors the determined second meta data.

The step of loading into the one or more processors the determined second meta data may be performed by the design module sending information about the second meta data to the one or more processors.

The method may comprise a step of displaying the first meta data and the second meta data together with the designed one or more virtual models of custom ear devices that the first meta data and the second meta data are associated with.

The one or more processors may be configured to display the first meta data and second meta data together with the associated designed one or more virtual models of custom ear devices in a graphical user interface. The graphical user interface may be of a software that may be located on a user's computer (e.g. an audiologist's computer), or may be located on a server, such as a software-as-a-service.

Displaying the first meta data and second meta data together with the associated designed one or more virtual models of custom ear devices in a graphical user interface, may allow a user, such as an audiologist or a person with hearing loss, to view the designed one or more virtual models of custom ear devices that are accepted as target products together with the associated first and second meta data, and may thus allow for a more efficient and comfortable selection of a target product (i.e. the target custom ear device).

The design module may be configured to send information about the designed accepted virtual models of custom ear devices. The design module may thus not necessarily send the designed accepted virtual models of custom ear devices, but only information about which combinations or which designed virtual models of custom ear devices may be accepted as target products, together with their associated second meta data.

This may allow reducing the load on a bandwidth between the design module and the one or more processors, and/or may allow reducing the load on the one or more processors handling a large amount of designed virtual models of custom ear devices.

The design module may thus be configured to send the designed accepted virtual models of custom ear devices with their associated second meta data directly to a user interface, such as a graphical user interface, and/or may allow the one or more processors to design the accepted combinations or accepted virtual models of custom ear devices, and send the accepted designed virtual models of custom ear devices with the first meta data and second meta data to a user interface, such as a graphical user interface.

The one or more processors may be configured to receive input from the design module indicative of one or more accepted virtual models of custom ear devices that may be accepted as target products based on geometric constraints.

The one or more processors may further be configured to display the one or more accepted virtual models of custom ear devices on a display.

According to the aspect, a computer-readable storage medium or computer program (e.g. software, a set of algorithms, etc.) is provided, that may comprise commands, which, when executed by a computer, may cause the computer to execute any of the methods described above.

The computer-readable storage medium may be any type of storage medium such as a CD, DVD, USB, hard-drive, memory chip, etc.

The computer program may be a software, a set of algorithms, etc.

The computer-readable storage medium or the computer program may be partly or fully located at a dispenser (i.e. audiologist) in a computer, such as a laptop, desktop computer, tablet, etc., or in a remote computer such as at a server. The computer program may be located partly or fully on a computer at a dispenser (i.e. audiologist) or in a remote server, such as providing a software-as-a-service.

According to the aspect, a system is provided for designing one or more virtual models of custom ear devices. The system may comprise one or more processors.

Custom ear devices may be custom hearing aid devices, custom in-ear monitors, custom earphones, custom ear molds, custom earshells, noise protection (e.g. for concerts, hunting, shooting, motor sports, etc.), custom sleep plugs, custom swim plugs, custom communication ear devices, custom radio earpieces (e.g. for news casters, security guards, etc.), or other similar types of custom ear devices having similar properties as the mentioned, which are to be designed based on a custom ear impression of a patient. Custom ear devices may comprise one or more of: custom hearing aid devices, custom in-ear monitors, custom earphones, or custom ear molds.

The one or more processors may comprise one processor, such as a CPU (central processing unit), with one or more processor cores. The one or more processors may comprise more than one processor, such as a plurality of CPUs, such as a processing cluster, wherein each of the plurality of CPUs includes one or more processor cores. The one or more processors may be located locally on a desktop computer, a laptop, a smartphone, a tablet, a smart tv, or a scanner, such as a handheld scanner. The one or more processors may partly or fully be located locally in one or more of the above-mentioned devices. The one or more processors may partly or fully be located in a server or a remote computer, that may be remotely relative to where the design and/or displaying of the one or more virtual models of custom ear devices is performed. The one or more processors may fully or partly be part of a cloud-computing service and may thus allow for using remote resources or may allow for a cloud-based processing.

The one or more processors may thus for example all or partly be located inside a desktop computer, or may all or partly be located remotely in a server as a cloud-based computing and operably connected to the desktop computer by cable, by a wireless network through a router, or Internet connection. The one or more processors may be distributed between two or more of the above mentioned locations. For example, some of the one or more processors may be located in the desktop computer in the form of a CPU (central processing unit), while some other of the one or more processors may be located in a remote server in a “cloud” as a cloud based computing service, and connected to the desktop computer via the internet.

The one or more processors may be configured to obtain ear impression data.

The ear impression data may be a virtual 3D model of an ear or part of an ear that has been scanned. The virtual 3D model may be obtained by scanning an ear or part of an ear using a handheld ear-scanner or by scanning an ear impression using a lab-scanner. The ear impression data may be 3D data, such as a 3D point cloud, or other scan data.

The one or more processors may be configured to obtain the ear impression data performing a direct scan using a handheld ear scanner or a lab-scanner, by loading from a memory unit, by loading from a file, by downloading from the internet, etc.

For example, an audiologist may take a physical ear impression of a person with hearing loss. The physical ear impression may subsequently be scanned using a lab-scanner, thus obtaining ear impression data. The obtained ear impression data may be used to create a virtual 3D model of the physical ear impression. The virtual 3D model may be created using a processor that is operably connected to the lab-scanner (e.g. a processor in the lab-scanner, a processor that is in a desktop computer connected to the lab-scanner, or a cloud-based processor that is connected to the lab-scanner using the internet). In another example, the obtained ear impression data may be received onto the audiologist's desktop computer (e.g. downloaded onto the audiologist's desktop computer from a server that is connected to the lab-scanner, received by e-mail, or otherwise received), and the virtual 3D model may be built on the audiologist's desktop computer, using a processor of the audiologist's desktop computer.

The one or more processors may be configured to receive one or more configuration requirements. The one or more configuration requirements may comprise one or more requirements for accepting a target product.

The one or more processors may be configured to receive the one or more configuration requirements as input from a user, such as a dispenser (audiologist), or as data from a file.

The configuration requirements may comprise one or more requirements for including, in a target product, one or more of: components, features, materials, size, retention, type, product, product series.

The one or more configuration requirements may comprise one or more requirements for including, in a target product, one or more of: type of custom ear device (e.g. behind the ear hearing aid device, in the ear hearing aid device, in the ear monitor, earphones, etc.), a shell type, a product family (e.g. a group of products of the same type, brand, or performance), a specific product (e.g. a specific product of a specific brand of a manufacturer or reseller), a preferred manufacturer, a preferred product, product series or product family from a manufacturer, components (e.g. speaker, speaker size, microphone, amplifier, battery, battery capacity, etc.), material (e.g. natural rubber, plastic, silicone, textile, etc.), features (e.g. options such as Bluetooth connection, buttons, improved noise cancelling, remote control, charging case (e.g. custom charging case), wireless charging, etc.), dimensions (e.g. thickness, size, etc.), retention (e.g. type of retention such as coverage of the ear, retention by friction in the ear, retention by retention means such as a hook/anchor behind the ear, etc.), performance (e.g. hearing loss compensation, noise cancellation, power consumption, etc.), software (e.g. software features e.g. noise cancellation, speech enhancement, etc.), and/or information such as price, colour, or hearing compensation information (e.g. audiogram), and ventilation information (e.g., small, medium, or large).

The configuration requirements may be one of the above or a combination of two or more of the above mentioned configuration requirements.

The above mentioned configuration requirements may be requirements that a user may require for a target product to be selected and/or accepted, and may thus be sent as a manufacturing order to be physically manufactured by a custom ear device manufacturer.

The target product may be a virtual model of a custom ear device, a set of specifications that describe a custom ear device, or a physically manufactured custom ear device, that meets the requirements of the one or more configuration requirements.

In an example, an audiologist, may in a graphical user interface of a software, displayed on the audiologist's desktop computer, select a set of configuration requirements comprising requests for a target product, based on a set of prerequisites of a person with hearing loss. The person with hearing loss may for example request a custom hearing aid that is of the type “in-the-ear hearing aid”, that is capable of compensating for a hearing loss of 20% of a normal human hearing, and that is capable of connecting to a smartphone using Bluetooth. The person with hearing loss may further require that the target product or an outer surface of the target product to be in a black colour, such that the target product is visually harder to detect when inserted inside the ear canal.

The one or more processors may be configured to access a datastore. The datastore may comprise component and/or material information. The component and/or material information may be associated with one or more components and/or materials of the target product. The component and/or material information may comprise a fixed value indicative of a fixed hierarchical order of the one or more components and/or material information.

The datastore may be a hard drive or a server. The datastore may be connected to servers, websites, or datastores, of one or more custom ear device manufacturers or manufacturers of components, materials, or software, for custom ear devices. The datastore may comprise a data storage such as a hard drive or a server, wherein data about products, components, features, materials, software, and information, from one or more ear device manufacturers are stored, and accessible by the one or more processors. The datastore may be a connection hub, providing or directing connection between the one or more processors and datastores or servers of one or more custom ear device manufacturers, wherein the datastores or servers of the custom ear device manufacturers comprise data or information of their respective products, components, features, materials, and software.

The access may be provided to the datastore by a network connection such as the Internet. The one or more processors may for example access the datastore using an internet connection, for example by connecting to a server of the datastore using the internet.

The datastore may comprise component and/or material information that may be associated with one or more components and/or materials of the target product. The components and/or materials may be products, such as hearing aid devices, custom hearing aid devices, in-ear monitors, custom in-ear monitors, earphones, custom earphones, ear molds, or custom ear molds; may be components, such as a part of the products, components of the products, microphones, speakers, amplifiers, microchips, batteries, Bluetooth connection, wireless antenna ; may be features, such as hearing loss compensation, noise cancellation, power consumption, speech enhancement, Bluetooth, wireless connection or communication, remote control; may be materials, such as polymeric materials, such as plastic, rubber, silicone, etc.; may be software, such as software that is configured to perform noise cancellation, speech enhancement, support wireless connection, support remote control, improve battery life, etc., and may be information related to the previously mentioned examples of components and/or materials, such as dimensions, properties, specifications, colour, price, etc.

The component and/or material information may be any information about, or data associated with, the components and/or materials. For example, the component and/or material information about the material and/or component “battery” may be type, brand, capacity, size, colour, which custom ear device it is compatible with, and/or price of the battery. Another example, the component and/or material information of a “product” may be: The amount of hearing loss the product can compensate, features (e.g. capable of Bluetooth connection), type of the retention (e.g. in-the-ear hearing aid), battery capacity, software that is included (e.g. comprising noise cancellation, speech enhancement, etc.), price, size, least venting hole size, etc.

The component and/or material information may comprise information that relates to one or more of: type of component, type of material, product, product series, features, component or material parameters, or performance.

The component and/or material information may comprise information associated with components and/or materials that may comprise geometric dimensions, such as lengths, widths, depths, and curvatures, and may further comprise virtual 3D models of the components and/or materials, and may further comprise information associated with properties such as material stiffness, temperature tolerances, etc.

The component and/or material information may be information and/or design rules that specify specific rules for designing virtual models of custom ear devices, such as e.g. that the manufacturer only allows 20 designs per order, that the manufacturer has specified a preferred prioritization for selecting a component or a product (e.g. for a specific product, material, or component, it is not allowed to print in a thickness that is less than 0.3 mm (millimeters), etc.).

The component and/or material information may comprise a fixed value. The fixed value may be any number. The component and/or material information may each, collectively, or in combination comprise a fixed value indicative of a fixed hierarchical order of the components and/or materials. The fixed value may be assigned to each individual component, material, component information, and/or material information in the datastore by the manufacturer.

For example, a fixed value may be assigned by a manufacturer to a particular product or a product information. A fixed value may be assigned to a particular product family or product series, thus the whole product family or product series having a fixed value collectively, or wherein each particular product in the product family or product series having the same fixed value. The fixed value may be indicative of a fixed hierarchical order of the one or more components and/or materials.

The fixed hierarchical order may be an expression of a prioritization of the fixed value of each of the component and/or material information or to each of the one or more components and/or materials, according to their value. The fixed hierarchical order may be the first order of each one of the component and/or material information or each one of the one or more components and/or materials, expressed, ranked, arranged, or ordered systematically according to their value.

The one or more processors may be configured to determine one or more combinations between the component and/or material information from the datastore and the one or more configuration requirements.

The one or more combinations may be a combination between each one of the component and/or material information from the datastore and each one of the one or more configuration requirements.

In a situation where no configuration requirement has been selected, the one or more combinations may be every custom ear device that is comprised of every one of the component and/or material information and every combination of every one of the component and/or material information.

In a situation where one or more configuration requirements have been selected, the one or more combinations may be every combination between each selected one or more configuration requirement and each component and/or material information. Thus, a first filtering may be performed, such that the custom ear devices may only be those who comprise component and/or material information that meet the requirements of the selected one or more configuration requirements.

The one or more processors may be configured to determine the one or more combinations for the component and/or material information that meet the one or more requirements of the one or more configuration requirements.

Determining the one or more combinations may be performed by the one or more processors. The one or more processors may be configured to obtain information related to one or more selected configuration requirements, and subsequently access the datastore, and determine one or more combinations of each component and/or material information, that meet the requirements of the one or more configuration requirements, such that one or more custom ear devices may be designed that is comprised of every combination of the components and/or materials of the component and/or material information that meet the requirements of the configuration requirements. Thus, in an embodiment, the one or more processors may be configured to determine the one or more combinations only for the component and/or material information that meet the one or more requirements of each of the one or more configuration requirements.

In an example, a configuration requirement may be specified wherein a custom ear device type is specified that is a hearing aid device which is capable of compensating a specific hearing loss, and which is capable of being manufactured in a specific size, that allows the hearing aid device to be retained in an ear canal of a specific person. The one or more processors may obtain information of the above mentioned specified configuration requirement, and subsequently access the datastore comprising the component and/or material information. The one or more processors may thus determine a set of combination, wherein every possible custom ear device is determined that may be built using every component and/or material of the database that is of a type “hearing aid device”, that is capable of compensating the specified hearing loss, and that is capable of being manufactured in the specified size.

The one or more processors may further be configured to assign a combined value to each of the one or more combinations. The combined value may be of a combined hierarchical order.

The combined value may be a combination of the respective fixed values of the components and/or material information.

The combined value may thus be the respective fixed value of the component and/or material information. For example, if no configuration requirement is specified, or a configuration requirement is specified that doesn't comprise a value, then the combined value may be the fixed value of the respective component and/or material information that is used in the particular combination comprising the particular combined value.

The one or more processors may be configured to design one or more virtual models of custom ear devices that may be based on the one or more combinations and the combined value.

The one or more processors may be configured to send a request to the design module for performing the design of the one or more virtual models of custom ear devices. The design module may be configured to receive a request from the one or more processor for designing the one or more virtual models of custom ear devices. The design module may be operably connected to the one or more processors. The design module may be a stand-alone software on a computer comprising the one or more processors, such as on an audiologist's desktop computer, or may be a software or a set of algorithms and/or neural networks located on a server, such as being a design service in the “cloud”.

Designing the one or more virtual models of custom ear devices may be performed by a design module. The design module may comprise one or more algorithms and/or one or more neural networks that are configured to design the one or more virtual models of custom ear devices based on an input from the one or more processors. The input from the one or more processors may be the one or more combinations or may be one or more instructions comprising information for designing the one or more virtual models of custom ear devices.

Designing the one or more virtual models of custom ear devices may be based on the one or more combinations and the combined value. The one or more processors may be configured to send instructions to the design module comprising information related to designing the one or more virtual models of custom ear devices of the one or more combinations. The instructions may comprise the combined values, and the design module may be configured to design the one or more virtual models of the custom ear devices in a sequence that is according to the combined values. The design module may be configured to send the designed one or more virtual models of custom ear devices to the one or more processors or directly to a graphical user interface, such as a graphical user interface of a computer comprising the one or more processors.

The design module is further described in more details in a later section.

The one or more configuration requirements may each, collectively, or in combination comprise an optional value indicative of an optional hierarchical order of the one or more configuration requirements. The optional value may be a number. The optional value may be assigned to each individual requirement of the one or more configuration requirements, to a set of requirements of the one or more configuration requirements, or to one configuration requirement of the one or more configuration requirements, by a user, such as a person with hearing loss or an audiologist. The optional value may be assigned on a website, such as a website for ordering custom ear devices, on a software, or other interfaces for inputting data.

The optional hierarchical order may be an expression of a prioritization of the optional value of each of the requirements of the one or more configuration requirements, or to one or more configuration requirements, according to their value. The optional hierarchical order may be the optional order of each requirement of the one or more configuration requirements, or of each one of the one or more configuration requirements, expressed, ranked, arranged, or ordered systematically according to their value.

In an example, an audiologist may, in a graphical user interface of a software displayed on the audiologist's desktop computer, select one or more configuration requirements, for example from a roll-down menu, and assign an optional value to each of the one or more configuration requirements.

In an example, an optional value may be assigned to each requirement in a configuration requirement individually. For example, the person with hearing loss may choose one configuration requirement that comprises a requirement wherein the type of custom hearing aid is an in-the-ear hearing aid, and may assign an optional value of 100 to that particular requirement, which may indicate a highest order or ranking in the hierarchy of the optional hierarchical order. The person with hearing loss may choose another requirement of the configuration requirement wherein the custom hearing aid is capable of connecting via a Bluetooth connection, and may assign an optional value of 90 to that particular requirement, indicating that the requirement of Bluetooth capability has a lower prioritization, order, or ranking, in the optional hierarchical order, than the requirement of the custom hearing aid being an in-the-ear hearing aid.

In another example, an optional value may be assigned to a set of requirements in a configuration requirement collectively. For example, the person with hearing loss may choose one configuration requirement that comprises a requirement wherein the type of custom hearing aid is an in-the-ear hearing aid, a requirement wherein the custom hearing aid is capable of connecting via a Bluetooth connection, and a requirement wherein a biodegradable material is used for an outer shell of the custom hearing aid. The person with hearing loss may subsequently assign an optional value of 100 to that particular configuration requirement, which may indicate a highest order or ranking in the hierarchy of the optional hierarchical order, and may thus indicate that this particular configuration requirement is the most preferred or most prioritized configuration requirement.

In an example where one or more configuration requirements have been selected, the one or more combinations may be assigned an optional value. An audiologist may for example select one or more configuration requirements on a graphical user interface, and assigning an optional value to each one of the one or more configuration requirements. The audiologist may for example select one configuration requirement wherein the custom ear device is of a type in-the-ear hearing aid, and assign an optional value of 100, and may select one configuration requirement wherein information is specified wherein a specific hearing loss is specified, and assign an optional value of 90, indicating that this configuration requirement is of a less priority than the configuration requirement comprising the optional value of 100. In another example, the audiologist may select a set of configuration requirements, and assign an optional value to the set of configuration requirements. The audiologist, may for example select a configuration requirement that the custom ear device is of a type in-the-ear hearing aid, may select a further configuration requirement of the type information, and select a specific hearing loss, and assign an optional value of 100 to the set of configuration requirement. The audiologist may select another set of configuration requirements, wherein the audiologist selects a configuration requirement wherein the type is of behind-the-ear hearing aid, a configuration requirement wherein the specific hearing loss is specified, and a configuration requirement wherein a feature is selected that allows for noise cancellation, and assign an optional value of 90 to the another set of configuration requirement, indicating that the another set of configuration requirement is of a less priority than the set of configuration requirement that has been assigned an optional value of 100.

In a situation where an optional value has been assigned to the one or more configuration requirements (i.e. the one or more configuration requirements comprising an optional value), the combined value may be a combination of the respective fixed value of the component and/or material information and the respective optional value of the one or more configuration requirements. Thus, the one or more configuration requirements may each comprise an optional value that may be indicative of an optional hierarchical order of the one or more configuration requirements. The combined value may combine the respective optional value and the respective fixed value.

The combined value may thus be a combination of the optional value of the one or more configuration information and the fixed value of the component and/or material information.

The one or more processors may be configured to determine the combined value by combining the fixed value and the optional value for each combination.

For example, a combination between a configuration requirement comprising an optional value of 100 and a component and/or material information comprising a fixed value of 80, comprises thus a combined value of 180.

For example, the design module designs virtual models of custom ear devices according to a sequence ordered hierarchically according to a combined value of each combination of one or more combinations determined by the one or more processors, wherein the combined values combine the optional values and the fixed values of the respective one or more combinations. The design module may for example first design a combination having a combined value of 200, then a combination having a combined value of 150, then a combination having a combined value of 100, etc.

In yet another example, an optional value may be assigned to a configuration requirement based on a combination of individual optional values of each requirement of the configuration requirement. For example, a configuration requirement comprising a requirement having an optional value of 50 and another requirement having an optional value of 70, the configuration requirement may thus have an optional value of 120.

The one or more processors may be configured to evaluate the combined value for each of the one or more combinations. The evaluation may be performed by reading the combined value of each of the one or more combinations. The one or more processors may be configured to detect the value of the combined value. The one or more processors may further be configured to determine a designing sequence that may be indicative of a hierarchically ordered sequence that may be based on the combined value.

The one or more processors may further be configured to feed into a design module the determined designing sequence. The design module may be configured to design the one or more virtual models of custom ear devices according to the designing sequence.

The one or more processors may be configured to feed the design module with the designing sequence, by sending the instructions of the design sequence to the design module. The design module may be configured to design the one or more virtual models of custom ear devices according to the designing sequence.

The designing sequence may be one or more instructions for designing one or more virtual models of custom ear devices in a particular order. The instructions may be configured to instruct a design module to design the one or more virtual models of custom ear devices in a particular order. The designing sequence may comprise information related to the one or more combinations and related to the respective combined values of the one or more combinations. The particular order may be based on the combined values of the one or more combinations. The one or more processors may be configured to determine the designing sequence based on the combined values of the one or more combinations, by evaluating the combined values of the one or more combinations.

For example, a designing sequence may comprise a set of instructions for designing virtual models of custom ear devices according to a combination A with a combined value of 200, a combination B with a combined value of 180, and a combination C with a combined value of 150, in that particular order, wherein the combination with the highest combined value is to be designed first. A design module will subsequently design combination A, then combination B, and then combination C.

The design module may be configured to determine whether each designed one or more virtual models of custom ear devices, is accepted as a target product. The design module may be configured to determine whether each designed one or more virtual models of custom ear devices is accepted as a target product, based on geometrical constraints of the one or more virtual models of custom ear devices.

Determining whether a designed virtual model of custom ear devices may be accepted as a target product or not, may allow reducing the risk of building a custom ear device that does not fit to the components that are included in the custom ear device, or to the ear of the person who is to wear the custom ear device, such as the ear of a person with hearing loss.

Furthermore, determining whether a designed virtual model of custom ear devices may be accepted as a target product or not, may allow for the reduction of the number of designed virtual models of custom ear devices that are sent to the one or more processors or to a graphical user interface, and a filtering of the possible target product may thus be performed.

Determining whether a designed virtual model of custom ear devices may be accepted as a target product may be performed by the design module. The design module may be configured to design one or more virtual models of custom ear devices, and may further be configured to subsequently determine whether the designed one or more virtual models of custom ear devices may be accepted as target products.

Determining whether the designed one or more virtual models of custom ear devices may be accepted as target products may be based on geometric constraints.

The geometric constraints may be that parts of the one or more virtual models of custom ear devices may not intersect (go through) parts of a virtual 3D model of a physical ear impression of a person. The design module may further determine, based on geometric dimensions of the components and/or materials that are comprised in the virtual model of custom ear devices, whether the components and/or materials intersect with the designed one or more virtual models of custom ear devices or not. Geometric constraints may further be constrains for preventing or reducing magnetic interference between components, such that some components may not be positioned too close to each other and not arranged in a specific direction or orientation that may increase or allow magnetic interference.

The design module may further be configured to evaluate the designed one or more virtual models of custom ear devices, by determining whether each of the one or more virtual models of custom ear devices can virtually accommodate the one or more component and/or materials which they are associated with.

The one or more processors may further be configured to receive the designed one or more virtual models of custom ear devices from the design module. The one or more processors may further be configured to display the designed one or more virtual models of custom ear devices on a display. Thus, the one or more processors may further be configured to display the received designed one or more virtual models of custom ear devices on a display.

The system may be configured to automatically suggest one or more custom ear devices. The one or more processors may thus be configured to perform an automatic product suggestion based on the specified one or more configuration requirements and based on the component and/or material information from the database. The automatic product suggestion may be performed using a product compare engine that may use matrix comparison.

The one or more processors may be configured to determine first meta data that relate to the performance of the one or more components and/or materials of the target product.

The first meta data may be data that may be predetermined by the manufacturers of the custom ear devices or manufacturers of components and materials of custom ear devices, and that may relate to preferences, performance, and or specifications, associated with each custom ear device, product, component, and material, such as e.g. a specific product allows for a Bluetooth connection, allows for a small venting hole, allows for improved hearing, etc. First meta data that is associated with a component, such as a battery, may for example be battery capacity or that a battery allows for wireless recharging, etc. The performance of the one or more components and/or materials of the target product may be preferences, performance, and or specifications, associated with each custom ear device, product, component, and material. The first meta data may further comprise information about size, price, etc.

The first meta data may be located in the datastore, and each component and/or material information may comprise one or more associated first meta data.

The design module may further be configured to determine second meta data that relates to the compatibility of the designed one or more virtual models of custom ear devices with components and in the ear.

The second meta data may be information that may be derived from the designed virtual models of the custom ear devices or from the designed virtual models of custom ear devices' interaction with the virtual 3D model of ear impression or virtual 3D models of components and or materials. The components may be components and materials of the component and/or material information from the datastore.

For example, the second meta data may be information relating to: retention in the ear, probability of occurring interference, size, placing, price, comfort inside the ear, etc.

Determining the second meta data may be performed by the design module. The design module may be configured to arrange each designed virtual model of custom ear devices into the virtual 3D model of the ear impression, and determine whether the arrangement is successful or acceptable, such that there is no intersection between any part of the virtual model of custom ear device and virtual 3D model of the ear impression. The design module may further arrange each of the virtual 3D models of components and/or materials of the component and/or material information that are present in a specific custom ear device or that have been specified by a user (e.g. audiologist) to be part of the target product, inside each virtual model of custom ear devices, and determine whether the arrangement is successful or acceptable, such that there is no intersection between any part of the virtual model of custom ear device and virtual 3D model of the components and/or materials.

The one or more processors may further be configured to receive the determined second meta data from the design module.

The design module may be configured to send information about the second meta data to the one or more processors.

The one or more processors may further be configured to display the first meta data and the second meta data together with the designed one or more virtual models of custom ear devices that each first meta data and second meta data are associated with.

The one or more processors may be configured to display the first meta data and second meta data together with the associated designed one or more virtual models of custom ear devices in a graphical user interface. The graphical user interface may be of a software that may be located on a user's computer (e.g. an audiologist's computer), or may be located on a server, such as a software-as-a-service.

Displaying the first meta data and second meta data together with the associated designed one or more virtual models of custom ear devices in a graphical user interface, may allow a user, such as an audiologist or a person with hearing loss, to view the designed one or more virtual models of custom ear devices that are accepted as target products together with the associated first and second meta data, and may thus allow for a more efficient and comfortable selection of a target product (i.e. the target custom ear device).

The design module may be configured to send information about the designed accepted virtual models of custom ear devices. The design module may thus not necessarily send the designed accepted virtual models of custom ear devices, but only information about which combinations or which designed virtual models of custom ear devices may be accepted as target products, together with their associated second meta data. This may allow reducing the load on a bandwidth between the design module and the one or more processors, and/or may allow reducing the load on the one or more processors handling a large amount of designed virtual models of custom ear devices.

The design module may thus be configured to send the designed accepted virtual models of custom ear devices with their associated second meta data directly to a user interface, such as a graphical user interface, and/or may allow the one or more processors to design the accepted combinations or accepted virtual models of custom ear devices, and send the accepted designed virtual models of custom ear devices with the first meta data and second meta data to a user interface, such as a graphical user interface.

The one or more processors may be configured to receive input from the design module indicative of one or more accepted virtual models of custom ear devices that may be accepted as target products based on geometric constraints.

The one or more processors may further be configured to display the one or more accepted virtual models of custom ear devices on a display.

The design module may include one or more algorithms for determining a custom ear device. The one or more algorithms may include a computer-implemented method for determining a custom ear device. The computer-implemented method may comprise loading into a processor an ear impression data; loading into the processor one or more configuration requirements for determining a custom ear device matching the ear impression data; receiving a user instruction causing the processor to load the ear impression data and the one or more configuration requirements into a neural network (NN), wherein the NN may be configured to output a final determined representation of the custom ear device comprising at least a set of operations as applied by the neural network, and wherein the set of operations may be complying with at least a part of the one or more configuration requirements. The method may further output the finally determined custom ear device and a final operations datafile that corresponds to the finally determined custom ear device.

The user may be able to change or modify the finally determined custom ear device, and in this example, the finally determined custom ear device is a partially determined custom ear device. The method may then further comprise receiving a user instruction causing the processor to perform changes to the at least one partially determined representation of the custom ear device in accordance with the user instruction, and to output: a finally determined custom ear device and an associated final operations datafile.

The neural network may be trained on the basis of a training data set comprising a plurality of training ear impression data; a plurality of target partial and/or fully determined representations of custom ear devices each complying with a plurality of target configuration requirements; and one or more target operations specific to the plurality of target partially and/or finally determined representations, and wherein the finally determined representations are complying with at least a part of the one or more target configuration requirements.

The final operations datafile may comprise production instructions configured to be utilized for manufacturing the finally constructed custom ear device.

The final operations datafile may comprise the set of operations as applied by the neural network in designing the partially constructed representation of the custom ear device, and a set of operations performed by the processor in accordance with the user instructions.

The method may comprise receiving a user instruction causing the processor to feed the changed partially constructed representation into at least one other neural network, wherein the at least one other neural network is configured to output an updated partially constructed representation of a custom ear device based on at least another set of operations, wherein the one other neural network is trained on the basis of a second training data set comprising at least a set of target operations corresponding to a specific partially and/or fully constructed representation.

The method may comprise receiving a user instruction causing the processor to feed the changed partially constructed representation into at least one algorithmic operation different from the set operations applied by the neural network, wherein the algorithmic operation is configured to output an updated partially constructed 3D model and/or the finally constructed representation.

The one or more configuration requirements may comprise a specified product type and design requirements for the specified product type as required by an audiologist and/or by a manufacture of custom ear devices through the configuration data.

The ear impression data may comprise a virtual 3D representation of the ear impression.

The set of operations may comprise one or one or more set of decision rules, one or more production data, wherein each of the one or more decision rules and/or production data provides instructions for production of the partially and/or fully constructed representation of a custom ear device as output from the neural network.

The method may comprise generating by the processor a 3D model of the ear impression data.

The one or more set of decision rules may comprise data representing operations applied by the neural network (NN) to construct the partially and/or finally constructed representation of the custom ear device to fit the anatomy of an ear canal of a user.

The one or more production data may comprise a plurality of production settings applied by the neural network (NN) to construct the partially and/or fully configured representation of the custom ear device.

The production data may comprise one or more of: a 3D representation of the electronic components to be arranged in the custom ear device; placement rules for the electronic components; and production settings defining device materials and sizes.

The one or more set of decision rules may comprise at least one or more set of operations representing: shaping a tip of the ear impression data, wherein the tip may be configured to face the inside of an ear canal when inserted therein; cutting a part of the ear impression data configured to face the outside of an ear canal when inserted therein; shaping the outer shape of the ear impression data; arrangement of the electronic components; casting for a soft silicone production of the digital ear impression placing of a sound bore; and placing of a vent canal.

The method may further comprise displaying, in a current view of a user interface, the finally constructed custom ear device with a representation of the final operations datafile; receiving, while in the current view of the user interface, a user input requesting that the at least one partially constructed representation of the custom ear device with a representation of the corresponding set of operations, may be displayed; and modifying, in response to the user input, the current view of the user interface to display the at least one partially constructed representation of the custom ear device with the representation of the corresponding set of operations.

The received user instruction may be configured as marking an area and/or pointing to an area on the fully constructed custom ear device, causing the processor to modify the current view of the user interface to display a partially constructed representation of the custom ear device corresponding to the set of operations applied to the marked and/or pointed at area.

The method may comprise receiving a user instruction causing the processor to perform changes to the partially constructed representation of the custom ear device in accordance with the user instruction, and to output an updated finally constructed ear custom device and an updated final operations datafile.

The method may further comprise means for executing the method as described above.

A further aspect of the disclosure is to obtain a system that may comprise a processor configured to receive one or more user instructions causing the processor to control the execution of a neural network engine comprising at least one neural network that is trained to output at least one partially and/or fully constructed representation of a custom ear device, a fast forward engine configured to receive the output from the neural network engine and to output at least one partially constructed representation of the custom ear device comprising at least a set of operations applied by the neural network and complying with at least a part of one or more configuration requirements; and an editing engine configured to receive the partially constructed representation of the custom ear device with the set of operations from the fast forward engine and to receive a user instruction representing changes applied to the partially constructed representation of the custom ear device.

The editing engine may be configured to receive a user instruction causing the processor to perform changes to the partially constructed 3D model in accordance with the user instruction, and to output a finally constructed custom ear device and a final operations datafile.

The editing engine may be configured to receive a user instruction causing the processor to perform changes to the partially constructed representation in accordance with the user instruction; output an updated partially constructed representation with an updated set of operations; and feeding the updated partially constructed representation into another neural network and/or an algorithmic operation.

The processor may be configured to be operatively connected with a storage engine, wherein the storage engine is configured to store thereon one or more ear impression data and one or more corresponding configuration requirements.

The storage engine may be configured as a cloud server configured to receive one or more ear impression data and corresponding one or more configuration requirements from one or more remote servers.

The storage engine may be configured to store thereon at least the finally constructed custom ear device and the final operations datafile.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an example system for designing and displaying virtual models of custom ear devices;

FIG. 2 illustrates a continuation of the example system illustrated in FIG. 1;

FIG. 3 shows a table with an overview of a method for designing and displaying virtual models of custom ear devices; and

FIG. 4 illustrates a processor configured to perform a computer implemented method according to examples of the disclosure.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

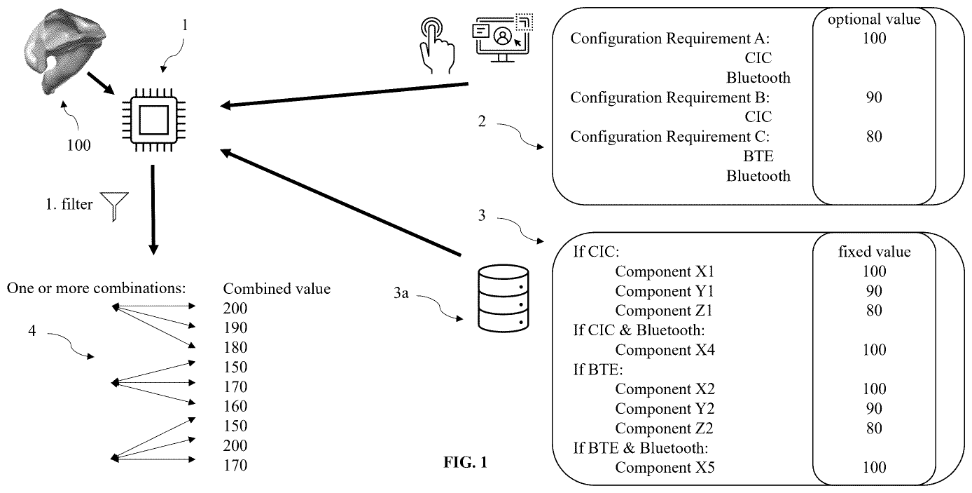

FIG. 1 illustrates an example system for designing and displaying virtual models of custom ear devices. The figure schematically illustrates the one or more processors 1 (shown as a central processing unit) and a virtual 3D model of a physical ear impression 100. An arrow between the virtual 3D model of the physical ear impression 100 and the one or more processors 1 illustrates that ear impression data related to the virtual 3D model of the physical ear impression 100 may be obtained by the one or more processors 1. Obtaining the ear impression data may occur by scanning a physical ear impression or by loading from a file.

The figure further illustrates one or more configuration requirements 2 (shown as three configuration requirements). In the illustrated example, the configuration requirements 2 comprise following requirements. Configuration requirement A comprises a requirement that the custom ear device must be a completely-in-canal hearing aid (CIC), and that the hearing aid must support a Bluetooth connection. The configuration requirement A is also shown comprising the optional value 100. In another example, the configuration requirement 2 may not comprise an optional value. In yet another example, each individual requirement of the configuration requirement 2 may comprise an individual optional value. In yet another example, where each individual requirement of the configuration requirement comprises an individual optional value, the particular configuration requirement 2 may comprise an optional value that is a combination of the individual requirements' individual optional values.

One configuration requirement B only comprises one requirement, which is that the type of custom ear device is a completely-in-canal hearing aid, and further comprises an optional value of 90. This indicates that the configuration requirement A has a higher ranking that the configuration requirement B, meaning that, if it is not possible to get a target product with the requirements of configuration requirement A, then the user may accept a target product with only the requirement of configuration requirement B.

One configuration requirement C is shown comprising a requirement that the type of custom ear device is a behind-the-ear hearing aid (BTE) and is capable of supporting a Bluetooth connection. The configuration requirement C further comprises an optional value of 80, which indicates, that the configuration requirement C has the lowest ranking amongst configuration requirements A, B, and C, and that a target product meeting the requirements of configuration requirement C is only accepted, if it is not possible to get a target product that meets the requirements of configuration requirements A or B.

A user, such as a dispenser (i.e. audiologist) may specify the configuration requirements, individual requirements, and optional values for each individual requirement and/or for each configuration requirement, via a user interface, such as a graphical user interface, in a software or website.

An arrow between the configuration requirements 2 and the one or more processors 1 illustrates that the configuration requirements 2 are loaded into the one or more processors 1.

FIG. 1 further illustrates a datastore 3a comprising component and/or material information 3. The component and/or material information 3 may comprise information associated with components or materials in a target product. The components or materials may be products, components, materials, features, software, rules from a manufacturer, etc. As an example, FIG. 1 illustrates, that if a configuration requirement is specified to require a CIC hearing aid type, component X1, Y1, Z1, may be selected. If a CIC hearing aid type that supports Bluetooth connection, the component X4 may be selected. If a BTE hearing aid type is required, then components X2, Y2, and Z2 may be selected. If a BTE hearing aid type is required, then component X5 may be selected. The figure further illustrates that each component and/or material information comprises a fixed value. The fixed value may be specified and assigned to each individual component and/or material information by a manufacturer. The component and/or material information may further comprise first meta data that comprise information relating to the performance of the components and materials of the component and/or material information 3. An arrow between the component and/or material information 3 or datastore 3a and the one or more processors 1 indicates, that the one or more processors 1 may be configured to access the datastore 3a and thus the component and/or material information 3.

The one or more processors 1 may subsequently determine one or more combinations 4 of the component and/or material information 3 that meet the requirements of the one or more configuration requirements 2. Hence, a first filtering may be performed. In another example, where no configuration requirements 2 are specified, the one or more processors 1 may determine combinations 4 of all available component and/or material information 3 from that are present in the datastore 3a. The one or more combinations 4 may comprise a combined value. In a situation where no configuration requirements 2 are specified or no optional values have been assigned to the configuration requirements 2, the combined value may be the fixed value of the component and/or material information 3 that the respective combination 4 is associated with. In a situation where optional values have been assigned to the one or more configuration requirements 2, the combined value may be a combination of the optional value and the fixed value of the respective configuration requirement 2 and the component and/or material information 3, that the respective combined value is associated with.

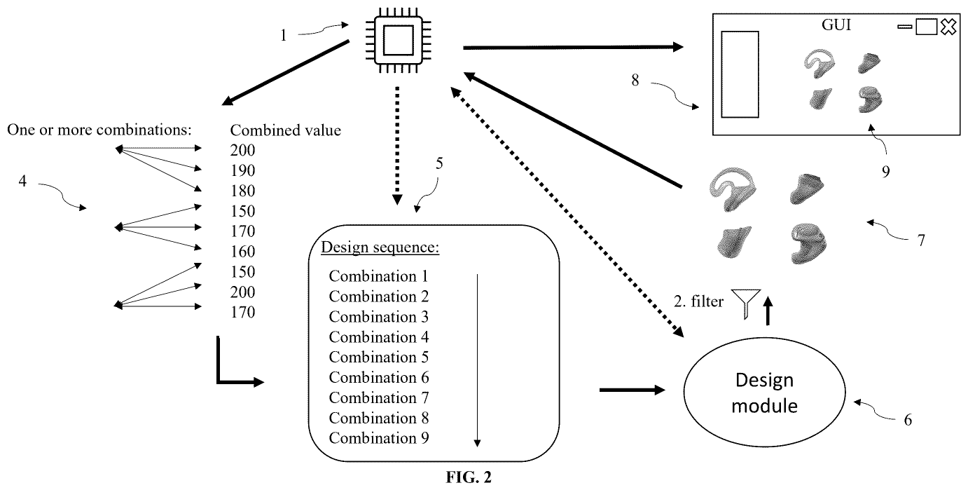

FIG. 2 illustrates a continuation of the example system illustrated in FIG. 1. The figure illustrates a designing sequence 5 comprising the one or more combinations 4 arranged in a hierarchical order. The hierarchical order may be based on a ranking of the one or more combinations 4. The ranking of the one or more combinations 4 may be based on the combined values, such as according to the combined value with the highest value, etc.

The designing sequence 5 is shown determined by the one or more processors 1, and based on the determined one or more combinations 4 and their respective combined values.

FIG. 2 further discloses a design module 6. The design module 6 may be configured to design the one or more combinations 4 that are specified in the designing sequence 5, in their respective order. The one or more processors 1 may be configured to send the designing sequence 5 to the design module 6. The design module 6 may be configured to design (virtually build) one or more virtual models of custom ear devices 7 based on the designing sequence 5.

FIG. 2 illustrates the designed one or more virtual models of custom ear devices 7 that the design module 6 has designed. The design module 6 may further be configured to determine second meta data based on geometric constraints of the designed one or more virtual models of custom ear devices 7. The second meta data may comprise information relating to the compatibility of the designed virtual models of custom ear devices 7 with the virtual 3D model of the physical ear impression 100, and/or with the components and/or materials of the component and/or material information 3.

The design module 6 may further be configured to determine whether a combination of the one or more combinations 4 from the designing sequence 5 may be accepted as a target product or not, based on the geometric constraints. Thus, a second filtering of target products may occur. The design module 6 may thus only send the designed one or more virtual models of custom ear devices 7 to the one or more processors 1 or to a user interface 9, such as a graphical user interface 9, that may be accepted as target products. The design module 6 may send the designed one or more virtual models of custom ear devices 7 together with the second meta data to the one or more processors 1.

FIG. 2 illustrates that the designed one or more virtual models of custom ear devices 7 that have been accepted as target products are sent to the one or more processors 1, together with the second meta data.

FIG. 2 illustrates, that the one or more processors is configured to send the designed one or more virtual models of custom ear devices 7 that have been accepted as target products and their associated first meta data and second meta data (illustrated collectively as meta data 9) to a graphical user interface 8. The user may thus view the designed one or more virtual models of custom ear devices that meet the requirements of the one or more configuration requirements 2 together with their associated meta data 9, and may thus select the possible target products.

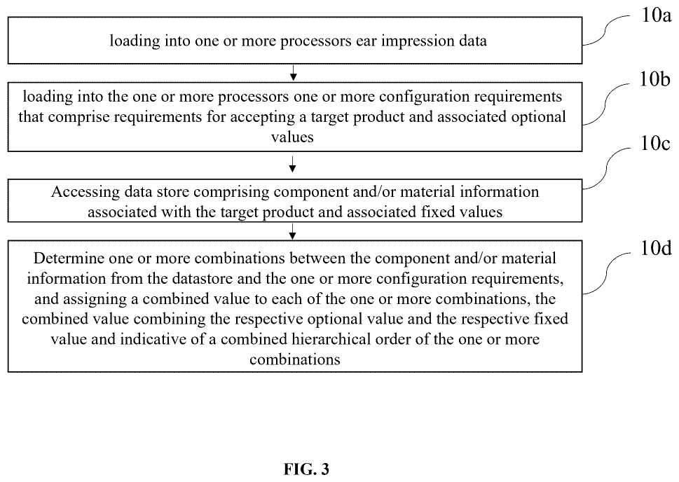

FIG. 3 shows a table with an overview of a method for designing virtual models of custom ear devices. The figure shows the method comprising a step 10a of loading into one or more processors 1 ear impression data 100. Loading the ear impression data 100 may be performed by performing a lab-scan of a physical ear impression or by loading the ear impression data 100 from a file.

FIG. 3 further illustrates a subsequent step 10b, wherein one or more configuration requirements 2 are loaded into the one or more processors 1. The one or more configuration requirements 2 may comprise requirements for accepting a target product and associated optional values. The optional values may be assigned by a user (e.g. a dispenser, audiologist, etc.), and may be indicative of a ranking of the requirements or of the one or more configuration requirements 2 (e.g. hierarchically ordered according a prioritization of the user).

FIG. 3 further illustrates a subsequent step 10c, wherein a datastore 3a comprising component and/or material information 3 is accessed. Accessing the datastore 3a may be performed by the one or more processors 1. The component and/or material information 3 may comprise information associated with a target product (e.g. designed virtual models of custom ear devices that are accepted based on the one or more configuration requirements 2 and based on geometric constraints). Each component and/or material information 3 may further comprise a fixed value.

FIG. 3 further illustrates a subsequent step 10c, wherein one or more combinations 4 between the component and/or material information 3 from the datastore 3a and the one or more configuration requirements 2 are determined. The step 10c may further comprise assigning a combined value to each of the one or more combinations 4. The combined value may combine the respective optional value and the respective fixed value, and may be indicative of a combined hierarchical order of the one or more combinations 4.

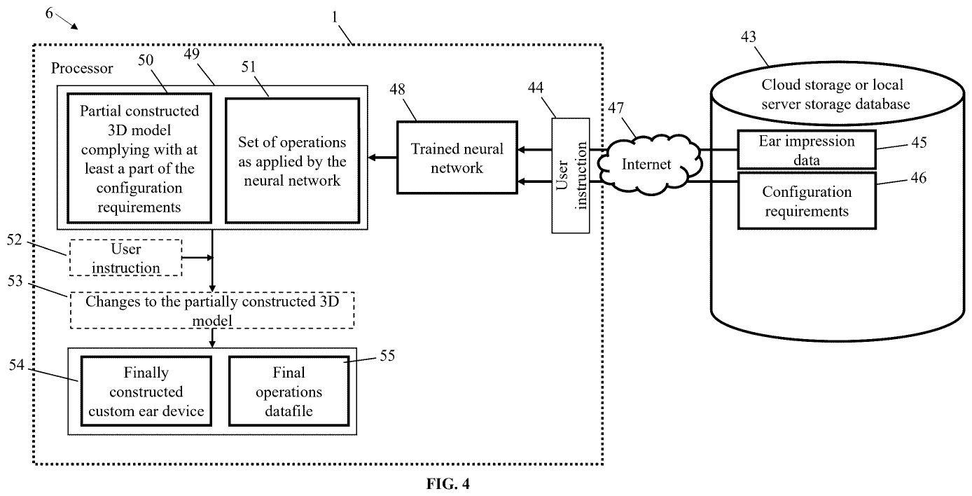

In more detail, the design module 6 is illustrated in FIG. 4. The design module may include one or more algorithms for determining a custom ear device. The one or more algorithms may include a computer-implemented method for determining a custom ear device. The computer-implemented method runs on the processor 1 that is configured to construct a custom ear device of a user based on data 43 input to the processor 1 upon receiving a user instruction 44 for loading the data 43 into the processor 1. The user instruction 44 may be any instruction given to the processor 1 through e.g. a user interface of a design application software. That is, the user instruction may e.g. be a click of a mouse, a touch on a screen and/or similar interaction with a graphical user interface of the design software application executing a method on the processor 1. Furthermore, ear impression data 45 is loaded into the processor 1. The ear impression data 45 can be obtained from a local storage unit (i. e a local server storage database) and/or from a cloud storage 43 as illustrated in FIG. 4.

Furthermore, the computer-implemented method is configured to load into the processor 1 one or more configuration requirements 46 for designing a custom ear device matching the ear impression data 45. The configuration requirements 46 may in correspondence with the ear impression data 45 be configured to be stored on a local storage unit (i.e. a local server storage database) and/or on a cloud storage 43. Both the ear impression data 45 and the configuration requirements 46 may be configured to be loaded into the processor 1 via a network, such as an internet 47, as illustrated in FIG. 4.

The processor 1 may be configured to access the datastore (not shown in FIG. 4) comprising component and/or material information associated with one or more components and/or materials of the target product, the component and/or material information comprising the fixed value indicative of the fixed hierarchical order of the one or more components and/or materials.

The loading of the ear impression data 45 and the configuration requirements 46 may be a result of a user instruction 44 applied to a user interface of design service application as described herein. Accordingly, the computer-implemented method is configured to receive a user instruction 44, causing the processor 1 to load the ear impression data 5 and the configuration data 46 into a neural network (NN) 48. The neural network 48 is configured to output 49 at least one partially constructed 3D model 50 of a custom ear device complying with at least a part of the one or more configuration requirements 46, and a set of operations 51 as applied by the neural network and complying with at least the part of the configuration requirements 46. The neural network output may comprise a set of operations and a 3D model of a partially constructed custom ear device. However, as already elaborated on, the neural network may be configured to only output the set of operations 51 and not necessarily to output the partial constructed 3D model, as such. That is, the set of operations coding for the operations to be applied to form a partial constructed model may be the main output form the neural network. That the neural network 48 described herein is configured to output at least a set of operations 51 coding for a partially constructed 3D model 50 allows a user (such as a professional modeler) to assess the partially constructed 3D model 50 as output from the neural network. That is, the neural network 48 is configured to output not only the finally constructed 3D model, which is also a possibility, but at least one or more partially constructed 3D models 50 which a user (such as the professional modeler) may then adjust and/or modify to create a more accurate and/or improved design of the custom ear device.

Alternatively, the computer-implemented method is further configured to receive a user instruction 52, causing the processor 1 to perform changes 53 to the partially constructed 3D model 50 in accordance with the user instruction 52. This allows the computer-implemented method to output a finally constructed ear custom device 54 and a final operations datafile 55, which contain information of the operations performed to design the finally constructed custom ear device 54 being by a neural network automatically and/or using further manually changes to e.g. adjust the partial designed custom ear devices output from the neural network 48.

By providing a computer-implemented method described above it is possible to provide a an automatically design of custom ear devices based on ear impression data and configuration requirements given by a user, while allowing an evaluation of the output from a neural network of any partial design steps that are needed in designing a custom ear device. The evaluation possibility that is provided for by the solution described herein, allows a user, such as a skilled professional modeler to adjust the design given by the neural network to more accurately reflect and comply with the requirements given, in case the neural network did not predict a partial design step optimally.

The design of custom ear devices by the design module 6 of FIG. 4 may additionally consider the one or more combinations 4 (FIG. 2) and the combined value, optionally in the design sequence of FIG. 2, in order to create the one or more virtual models of custom ear devices that are manufacturable, that fit the specific ear geometry of the patient and optionally in a hierarchical order.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/ unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to a component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e., to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element, but an intervening element may also be present, unless expressly stated otherwise. Furthermore, “connected” or “coupled” as used herein may include wirelessly connected or coupled. As used herein, the term “and/or” includes any and all combinations of one or more of the associated listed items. The step of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. A computer-implemented method for designing one or more virtual models of custom ear devices, the method comprising:
loading into one or more processors ear impression data,
loading into the one or more processors one or more configuration requirements that comprise one or more requirements for accepting a target product,
providing access for the one or more processors to a datastore comprising component and/or material information associated with one or more components and/or materials of the target product, the component and/or material information comprising a fixed value indicative of a fixed hierarchical order of the one or more components and/or materials, determining, using the one or more processors, one or more combinations between the component and/or material information from the datastore and the one or more configuration requirements, and assigning a combined value of a combined hierarchical order to each of the one or more combinations, wherein the combined value comprises at least the respective fixed value of the component and/or material information,
designing one or more virtual models of custom ear devices based on the one or more combinations and the combined value,
selecting the target product from the one or more designed virtual models of custom ear devices such that the target product fits the ear impression data, and generating a control file for a 3D printer, wherein the control file comprises instructions for manufacturing of the target product.

2. The method according to claim 1, wherein selecting the target product from the one or more designed virtual models of custom ear devices is based on geometric constraints of the one or more virtual models of custom ear devices.

3. The method according to claim 2, wherein the geometric constraints comprise constraints for arranging components to prevent or reduce magnetic interference.

4. The method according to claim 1, wherein the target product is a shell with impression of components on the inside surface of the shell.

5. The method according to claim 1, wherein:
the one or more configuration requirements each comprises an optional value indicative of an optional hierarchical order of the one or more configuration requirements, and
wherein the combined value combines the respective optional value and the respective fixed value.

6. The method according to claim 1,
wherein the step of determining the one or more combinations is performed for the component and/or material information that meet the one or more requirements of each of the one or more configuration requirements.

7. The method according to claim 1, further comprising performing first filtering such that the designed one or more virtual models of custom ear devices each comprise the component and/or material information that meet the requirements of the one or more configuration requirements.

8. The method according to claim 1, wherein the method further comprises the steps of:
evaluating the combined value for each of the one or more combinations and determining a designing sequence indicative of a hierarchically ordered sequence based on the combined value, and
feeding into a design module the determined designing sequence; wherein the design module is configured to design the one or more virtual models of custom ear devices according to the designing sequence.

9. The method according to claim 1, wherein the method further comprises the steps of:
loading into the one or more processors the designed one or more virtual models of custom ear devices from the design module, and
displaying the designed one or more virtual models of custom ear devices on a display.

10. The method according to claim 1, wherein the method further comprises the steps of:
determining, using the one or more processors, first meta data that relate to the performance of the one or more components and/or materials of the target product,
determining, using the design module, second meta data that relate to the compatibility of the designed one or more virtual models of custom ear devices with components and in the ear, and
loading into the one or more processors the determined second meta data.

11. The method according to claim 10, wherein the method further comprises the step of displaying the first meta data and the second meta data together with the designed one or more virtual models of custom ear devices that the first meta data and the second meta data are associated with.

12. The method according to claim 1, wherein:
the component and/or material information comprise information that relate to one or more of: type of component, type of material, product, product series, features, or performance,
the custom ear devices comprise one or more of: custom hearing aid devices, custom in-ear monitors, custom earphones, or custom ear molds, and
the one or more configuration requirements comprise one or more requirements for including, in a target product, one or more of: components, features, materials, size, retention, type, product, product series.

13. The method according to claim 1, further comprising manufacturing the target product based on the generated control file.

14. A computer-readable storage medium comprising commands, which, when executed by a computer, causes the computer to execute the method according to claim 1.

15. A computer program product comprising commands, which, when executed by a computer, cause the computer to execute the method according to claim 1.

16. A system for designing one or more virtual models of custom ear devices, the system comprising:
one or more processors configured to:
obtain ear impression data,
receive one or more configuration requirements that comprise one or more requirements for accepting a target product,
access a datastore comprising component and/or material information associated with one or more components and/or materials of the target product, the component and/or material information comprising a fixed value indicative of a fixed hierarchical order of the one or more components and/or material information,
determine one or more combinations between the component and/or material information from the datastore and the one or more configuration requirements, and assign a combined value of a combined hierarchical order to each of the one or more combinations, wherein the combined value comprises at least the fixed value of the component and/or material information,
design one or more virtual models of custom ear devices based on the one or more combinations and the combined value,
select the target product from the one or more designed virtual models of custom ear devices such that the target product fits the ear impression data, and
generate a control file for a 3D printer, wherein the control file comprises instructions for production of the target product.

17. The system according to claim 16, wherein:
the one or more configuration requirements each comprising an optional value indicative of an optional hierarchical order of the one or more configuration requirements, and
wherein the combined value combines the respective optional value and the respective fixed value.

18. The system according to claim 16, wherein the one or more processors is configured to determine the one or more combinations for the component and/or material information that meet the one or more requirements of the one or more configuration requirements.

19. The system according to claim 16, wherein the one or more processors is further configured to:
evaluate the combined value for each of the one or more combinations, by reading the combined value of each of the one or more combinations, and determine a designing sequence indicative of a hierarchically ordered sequence based on the combined value, and
feed into a design module the determined designing sequence; wherein the design module is configured to design the one or more virtual models of custom ear devices according to the designing sequence.

20. The system according to claim 19, wherein the design module is further configured to determine whether each designed one or more virtual models of custom ear devices, is accepted as a target product, based on geometrical constraints of the one or more virtual models of custom ear devices.
